(19) United States
(12) Patent Application Publication
(10) Pub. No.: US 2014/0271764 A1
(43) Pub. Date: Sep. 18, 2014

(54) Bioerodible Silicon-Based Delivery Vehicles for Delivery of Therapeutic Agents

(71) Applicant: pSivida US, Inc., Watertown, MA (US)

(72) Inventors: Paul Ashton, Newton, MA (US); Gerard Riedel, Concord, MA (US); Hong Guo, Wayland, MA (US)

(73) Assignee: pSivida US, Inc., Watertown, MA (US)

(21) Appl. No.: 14/206,843

(22) Filed: Mar. 12, 2014

Related U.S. Application Data
(60) Provisional application No. 61/778,121, filed on Mar. 12, 2013.

(51) Int. Cl. A61K 47/02; A61K 38/35

(52) U.S. Cl. CPC A61K 47/02; A61K 38/35
USPC 424/422; 514/10.8

(57) ABSTRACT

This invention discloses bioerodible delivery compositions for delivering peptide therapeutic agents. The delivery compositions comprise a porous silicon-based carrier material loaded with the therapeutic agent. The delivery compositions may be used in vitro or in vivo to deliver the therapeutic agent, preferably in a controlled fashion over an intended period of time such as over multiple days, weeks or months. The delivery compositions may be used for treating or preventing conditions of a patient such as chronic diseases.

### CROSS-REFERENCE TO RELATED APPLICATIONS

This application claims the benefit of U.S. Provisional Application No. 61/778,121 filed on Mar. 12, 2013; the entire content of said application is incorporated herein in its entirety by this reference.

### BACKGROUND

There has been considerable interest within the pharmaceutical industry in the development of dosage forms which provide controlled release of therapeutic agents over a period of time. Releasing an active substance in this way can help to improve bioavailability and ensure that appropriate concentrations of the agent are provided for a sustained period without the need for repeated dosing. In turn, this also helps to minimize the effects of patient non-compliance which is frequently an issue with other forms of administration.

Some known delivery vehicles provide active ingredients that are incorporated into polymer and sol-gel systems by entrapment during synthesis of the matrix phase. Microencapsulation techniques for biodegradable polymers include such methods as film casting, molding, spray drying, extrusion, melt dispersion, interfacial deposition, phase separation by emulsification and solvent evaporation, air suspension coating, pan coating and in-situ polymerization. Melt dispersion techniques are described, for example, in U.S. Pat. No. 5,807,574 and U.S. Pat. No. 5,665,428.

In an alternative approach, the active ingredient is loaded after formation of the porous matrix is complete. Such carrier systems generally have micron-sized rather than nanometer-sized pores to allow the agents to enter into the pores. U.S. Pat. No. 6,238,705, for example, describes the loading of macroporous polymer compositions by simple soaking in a solution of the active ingredient and U.S. Pat. Nos. 5,665,114 and 6,521,284 disclose the use of pressure to load the pores of implantable prostheses made of polytetrafluoroethene (PTFE). While this approach may be effective for small organic molecules, larger molecules such as proteins tend to aggregate in large pores and do not effectively release in vivo in a controlled manner.

With smaller pores, it has proved difficult to incorporate high concentrations of therapeutic agents due to blocking of the narrow pores. Deposition of material towards the opening of the pores tends to prevent a high proportion of the material from occupying the pore system. The problem of achieving high loading of the active ingredient limits the effectiveness of many currently known delivery systems.

Another concern when delivering therapeutic agents through an implant is the biocompatibility of the implant following release of the drug. Bioerodible or resorbable implant materials would be an attractive alternative to implants that require removal following release of the drug. The design and preparation of bioerodible implants for carrying therapeutic agents has begun to be explored. US Publication No. 20120177695 describes a drug delivery system comprising a porous silicon material.

Therefore, there remains a continuing need for the development of improved dosage forms for the controlled release of therapeutic agents, which are biocompatible and are capable of delivering biomolecules in a sustained fashion.

### SUMMARY

Disclosed are bioerodible compositions, such as implants, for delivering peptide therapeutic agents in a controlled manner. The compositions comprise a porous silicon-based carrier material loaded with the therapeutic agent. The compositions may be used in vitro or in vivo to deliver the therapeutic agent, preferably in a controlled fashion over an intended period of time such as over multiple days, weeks or months. The carrier material is preferably formed from a bioerodible or resorbable material, e.g., a silicon-based material such as elemental silicon or silicon dioxide, such that removal following release of the therapeutic agent is unnecessary. In certain such embodiments, the carrier material and its breakdown products are biocompatible such that the biological side effects from the bioerosion of the carrier material are minimal or innocuous.

In certain embodiments, the carrier material comprises porous silicon dioxide, such as mesoporous silicon dioxide or amorphous silica, such as fumed silica. The average pore size of the carrier material is typically selected so that it may carry the therapeutic agent, and example pore sizes are from 2-50 nm in diameter, such as from about 5 to about 40 nm in diameter, from about 15 to about 40 nm in diameter, from about 20 to about 30 nm in diameter, from about 2 to about 15 nm in diameter, or about 5 to about 10 nm in diameter.

In certain embodiments, the therapeutic agent is a peptide with a molecular weight between about 1,000 amu and about 10,000 amu, and may be about 1,000 to about 5,000 amu, between about 2,000 and about 5,000 amu, between about 3,000 and about 5,000 amu or between about 4,000 and about 5,000 amu.

The size of a therapeutic agent may alternatively be characterized by the molecular radius, which may be determined, for example, through X-ray crystallographic analysis or by hydrodynamic radius. The therapeutic agent may be a peptide, e.g., with a molecular radius selected from 0.5 nm to 20 nm, such as about 0.5 nm to 10 nm, even from about 1 to 8 nm. Preferably, a suitable pore radius to allow access to particular agents, e.g., peptides, is selected according to a pore-therapeutic agent (agent) differential, defined herein as the difference between the radius of an agent and a radius of a pore. For example, the pore-agent differential for insulin, with a hydrodynamic radius of 1.3 nm and a pore with a minimum radius of 4.8 nm has a pore-protein differential of 3.5 nm. A pore-agent differential may be used to determine minimum suitable average pore size for accommodating a peptide of a particular radius. The pore-peptide differential may typically be selected from about 3.0 to about 5.0 nm.

Typically, the carrier materials are selected to have an average pore size to accommodate the therapeutic agent. The average pore size of the carrier material may be chosen based on the molecular weight or the molecular radius of the therapeutic agent to be loaded into the pores of the carrier material. For example, a therapeutic agent of molecular weight selected from about 1,000 amu to about 10,000 amu, and maybe about 1,000 amu to about 5,000 amu, from about 2,000 amu to about 5,000 amu, from about 3,000 amu to about 5,000 amu or from about 4,000 amu to about 5,000 amu may be used with a carrier material of larger average pore size such as from about 1 nm to about 40 nm. In certain embodiments, a therapeutic agent of molecular weight selected from 1,000 amu to 5,000 amu may be used with a carrier material of smaller average pore size such as from about 1 nm to about 10 nm.

In certain embodiments, the compositions are prepared by forming the porous carrier material first and then loading the pores with the therapeutic agent.

The invention includes methods for loading a therapeutic agent into the pore of a porous silicon-based carrier material, comprising contacting a porous silicon-based carrier material with a therapeutic agent. One exemplary method for loading a therapeutic agent into the pore of a porous silicon-based carrier material comprises selecting a porous silicon-based carrier having pore sizes dimensionally adapted to allow a single peptide to load into the pore such that opposite sides of the peptide engage opposite sides of the pore. One method for loading a therapeutic agent into the pore of a porous silicon-based carrier material comprises selecting a porous silicon-based carrier having pore sizes dimensionally adapted to admit only a single agent into the width of a single pore at one time (i.e., longitudinal series along the length of a pore are not excluded), e.g., two agents could not be accommodated if positioned side-by-side (laterally) within a pore.

The compositions may be disposed on the skin or on the surface of the eye. Alternatively, the compositions may be disposed within the body of a mammal, such as within the eye of a patient, or within any other tissue or organ of the patient's body. In particular applications, the composition is disposed subcutaneously, intramuscularly, subconjunctivally or in the vitreous of the eye. The composition may be used for treating or preventing conditions of a patient such as chronic diseases. In certain embodiments, the compositions are for treating or preventing diseases of the eye such as glaucoma, macular degeneration, diabetic macular edema and age-related macular degeneration. The therapeutic agent may release in a controlled manner over a period of days, weeks or months, for example, to treat or prevent diseases of the eye such as macular degeneration.

The invention comprises stabilized formulations and methods of stabilizing therapeutic agents in a porous carrier material as described herein. In certain embodiments, the invention comprises stabilizing peptides in the pores of the carrier material such that the half-life or the shelf life of the peptide is superior to the half-life or shelf life of the peptide outside of the carrier material.

In certain embodiments, the invention provides a sustained release drug delivery composition comprising:

a) a carrier material comprising a silicon-based compound; and b) at least one therapeutic agent associated with the carrier material, wherein the at least one therapeutic agent includes adrenocorticotropic hormone (ACTH) or an analog thereof.

The invention further includes a syringe comprising a composition of porous silicon-based carrier material, wherein the composition comprises less than 2% biomolecules. The syringes may be used to administer a therapeutic agent, such as a peptide, by: a. providing a syringe preloaded with a porous silicon-based carrier material; b. contacting the carrier material with a therapeutic agent; and c. administering the carrier material to the patient. Step b may be carried out by drawing the therapeutic agent into the syringe. Between steps b and c, an incubation time, e.g., 10 min, 20 min or 30 min, may be taken to allow the therapeutic agent to adsorb into the pores of the carrier material.

### BRIEF DESCRIPTION OF THE DRAWINGS

The devices will now be described in more detail with reference to preferred embodiments, given only by way of example, and with reference to the accompanying drawings, in which:

FIG. 1 is a cumulative in vitro release profile of oxidized anodized silicon particles loaded with ACTH (carrier:ACTH 10:1 w/w) in PBS at 37° C. over 7 days.

### DETAILED DESCRIPTION

### Overview

The invention comprises a sustained release drug delivery composition comprising: a) a carrier material comprising a silicon-based compound; and b) at least one therapeutic agent associated with the carrier material, wherein the at least one therapeutic agent includes a peptide, such as adrenocorticotropic hormone (ACTH) or an analog thereof. In some embodiments, the at least one therapeutic agent includes an ACTH analog selected from corticotropin, tetracosactide or cosyntropin. In some embodiments, the composition comprises particles of carrier material that are sized for injection through a needle. In some embodiments, the silicon-based compound of the carrier material comprises one or more of: porous silicon, polycrystalline silicon, synthetic amorphous silica, and resorbable or bio-erodible silicon. In some embodiments, the porous silicon is mesoporous. In some embodiments, the porous silicon-based compound is amorphous silica, such as fumed silica. In some embodiments, the silicon-based compound has a silica or silicon oxide surface. In some embodiments, the silicon-based compound comprises pores that are substantially parallel.

Sustained and controlled delivery of therapeutic agents to patients, particularly patients with chronic conditions such as ophthalmic diseases, glaucoma, keratitis, iritis, iridocyclitis, diffuse posterior uveitis and choroiditis, optic neuritis, chorioretinitis, anterior segment inflammation, multiple sclerosis, infantile spasms, rheumatic disorders, psoriatic arthritis, rheumatoid arthritis, including juvenile rheumatoid arthritis (selected cases may require low-dose maintenance therapy), ankylosing spondylitis, collagen diseases, systemic lupus erythematosus, systemic dermatomyositis (polymyositis), dermatological diseases, severe erythema multiforme, Stevens-Johnson syndrome or cancer, allergic states, serum sickness, respiratory diseases, symptomatic sarcoidosis, edematous state, proteinuria, nephrotic syndrome, is becoming increasingly important in modern medical therapy. Many therapies are most effective when administered at frequent intervals to maintain a near constant presence of the active agent within the body. While frequent administration may be recommended, the inconvenience and associated difficulty of patient compliance may effectively prevent treatment in this manner. As a result, sustained release compositions that release therapeutic agents in a controlled manner are very attractive in fields such as cancer therapy and treatment of other chronic diseases. Furthermore, sustained release compositions may allow for dose reduction of the therapeutic agent, thereby leading to reduced side effects.

Compositions that release therapeutic agents in vivo or in vitro may be formed from a variety of biocompatible or at least substantially biocompatible materials. One type of composition employs a silicon-based carrier material. Silicon-based carrier materials may include, for example, elemental silicon, and oxidized silicon in forms such as silicon dioxide (silica), or silicates. Some silicon-based materials have demonstrated high biocompatibility and beneficial degradation in biological systems, eliminating the need to remove the material following release of the therapeutic agent.

Tests show that high porosity silicon-based materials, e.g., 80% porosity, are resorbed faster than medium porosity silicon-based material, e.g., 50% porosity, which in turn is resorbed faster than bulk silicon-based material, which shows little to no sign of bioerosion or resorption in biological systems. Furthermore, it is understood that the average pore size of the carrier material will affect the rate of resorption. By adjusting the average pore size of a carrier material as well as the porosity of the material, the rate of bioerosion may be tuned and selected. The rate of erosion of the silicon can be controlled by controlling the porosity (higher porosity materials are corroded faster) and the pore size (smaller pores for same porosity are corroded faster), and the barrier thickness.

Silicon-based materials are often prepared using high temperatures and organic solvents or acidic media to form the porous material and load the therapeutic agent within the pores. These conditions may be suitable for certain molecules such as salts, elements, and certain highly stable small organic molecules. However, for loading large organic molecules, such as proteins or antibodies, caustic and/or severe conditions during the preparation or loading of the template could lead to denaturing and deactivation, if not complete degradation of the active agent. Loading large molecules such as antibodies into the carrier material under mild conditions is a feature of the methods described herein that is particularly advantageous for large organic molecules such as proteins.

The particle size of the silicon-based carrier material may also affect the rate at which the pores of the carrier material may be loaded with the therapeutic agent. Smaller particles, e.g., particles in which the largest diameter is 20 microns or less, may load more rapidly than particles in which the largest diameter is greater than 20 microns. This is particularly apparent when the pore diameters are similar in dimensions to the molecular diameters or size of the therapeutic agents. The rapid loading of smaller particles may be attributed to the shorter average pore depth that the therapeutic agent must penetrate in smaller particles and the increased surface area.

### DEFINITIONS

As used herein the specification, “a” or “an” may mean one or more. As used herein in the claim(s), when used in conjunction with the word “comprising”, the words “a” or “an” may mean one or more than one. As used herein “another” may mean at least a second or more.

Bioerode or bioerosion, as used herein, refers to the gradual disintegration or breakdown of a structure or enclosure over a period of time in a biological system, e.g., by one or more physical or chemical degradative processes, for example, enzymatic action, hydrolysis, ion exchange, or dissolution by solubilization, emulsion formation, or micelle formation.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

Resorption or resorbing as used herein refers to the erosion of a material when introduced into or onto a physiological organ, tissue, or fluid of a living human or animal.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

Unless otherwise indicated, the term peptide refers to molecules comprising peptide bonds, such as molecules built from the 20 amino acids used in natural mammalian protein synthesis and/or analogs thereof, that have molecular weights equal to or greater than 1000 amu, preferably greater than 2000 amu, or even greater than 3000 amu, up to 10,000 amu. Unless otherwise indicated, a small molecule therapeutic molecule refers to a molecule with a molecular weight less than 1000 amu.

Silicon-Based Materials and Other Bioerodible Carriers

The compositions and methods described herein provide, among other things, compositions comprising a porous silicon-based carrier material wherein at least one peptide therapeutic agent is disposed in a pore or otherwise adsorbed to a surface of the carrier material.

The described methods use such devices for treatment or prevention of diseases, particularly ophthalmic diseases, glaucoma, keratitis, iritis, iridocyclitis, diffuse posterior uveitis and choroiditis, optic neuritis, chorioretinitis, anterior segment inflammation, multiple sclerosis, infantile spasms, rheumatic disorders, psoriatic arthritis, rheumatoid arthritis, including juvenile rheumatoid arthritis (selected cases may require low-dose maintenance therapy), ankylosing spondylitis, collagen diseases, systemic lupus erythematosus, systemic dermatomyositis (polymyositis), dermatological diseases, severe erythema multiforme, Stevens-Johnson syndrome or cancer, allergic states, serum sickness, respiratory diseases, symptomatic sarcoidosis, edematous state, proteinuria, nephrotic syndrome.

Furthermore, the described methods of preparing devices provide compositions which are characterized by sustained and controlled release of peptide therapeutic agents, such as ACTH tetracosactide, cosyntropin, or corticotropin.

The carrier material typically comprises a silicon-based carrier material such as elemental silicon, silicon dioxide (silica), silicon monoxide, silicates (compounds containing a silicon-bearing anion, e.g., SiF₆²⁻, Si₂O₇⁶⁻, or SiO₄⁴⁻), or any combination of such materials. In certain embodiments, the carrier material comprises a complete or partial framework of elemental silicon and that framework is substantially or fully covered by a silicon dioxide surface layer. In other embodiments, the carrier material is entirely or substantially entirely silica.

Although silicon-based materials are preferred carrier materials for use in the present invention, additional bioerodible materials with certain common properties (e.g., porosity, pore size, particle size, surface characteristics, bioerodibility, and resorbability) as the silicon-based materials described herein may be used in the present invention. Examples of additional materials that may be used as porous carrier materials are bioerodible ceramics, bioerodible metal oxides, bioerodible semiconductors, bone phosphate, phosphates of calcium (e.g., hydroxyapatite), other inorganic phosphates, carbon black, carbonates, sulfates, aluminates, borates, aluminosilicates, magnesium oxide, calcium oxide, iron oxides, zirconium oxides, titanium oxides, and other comparable materials.

In certain embodiments, the carrier material comprises silica, such as greater than about 50% silica, greater than about 60 wt % silica, greater than about 70 wt % silica, greater than about 80 wt % silica, greater than about 90 wt % silica, greater than about 95 wt % silica, greater than 99 wt % silica, or even greater than 99.9 wt % silica. Porous silica may be purchased from suppliers such as Davisil, Silicycle, and Macherey-Nagel.

In certain embodiments, the carrier material comprises elemental silicon, greater than 60 wt % silicon, greater than 70 wt % silicon, greater than 80 wt % silicon, greater than 90 wt % silicon, or even greater than 95 wt % silicon. Silicon may be purchased from suppliers such as Vesta Ceramics.

Purity of the silicon-based material can be quantitatively assessed using techniques such as Energy Dispersive X-ray Analysis, X-ray fluorescence, Inductively Coupled Optical Emission Spectroscopy or Glow Discharge Mass Spectroscopy.

The carrier material may comprise other components such as metals, salts, minerals or polymers. The carrier material may have a coating disposed on at least a portion of the surface, e.g., to improve biocompatibility of the device and/or affect release kinetics.

The silicon-based carrier material may comprise elemental silicon or compounds thereof, e.g., silicon dioxide or silicates, in an amorphous form. In certain embodiments, the elemental silicon or compounds thereof is present in a crystalline form. In other embodiments, the carrier material comprises amorphous silica and/or amorphous silicon. In certain embodiments, the silicon-based material is greater than about 60 wt % amorphous, greater than about 70 wt % amorphous, greater than about 80 wt % amorphous, greater than about 90 wt % amorphous, greater than about 92 wt % amorphous, greater than about 95 wt % amorphous, greater than about 99 wt % amorphous, or even greater than 99.9 wt % amorphous. In certain embodiments, the amorphous silicon-based compound is fumed silica. In certain embodiments, the amorphous silicon-based compound is synthetic amorphous silica.

X-ray diffraction analysis can be used to identify crystalline phases of silicon-based material. Powder diffraction can be taken, for example, on a Scintag PAD-X diffractometer, e.g., equipped with a liquid nitrogen cooled germanium solid state detector using Cu K-alpha radiation.

The silicon-based material may have a porosity of about 40% to about 95% such as about 60% to about 80%. Porosity, as used herein, is a measure of the void spaces in a material, and is a fraction of the volume of voids over the total volume of the material. In certain embodiments, the carrier material has a porosity of at least about 10%, at least about 20%, at least about 30%, at least about 40%, at least about 50%, at least about 60%, at least about 70%, at least about 80%, or even at least about 90%. In particular embodiments, the porosity is greater than about 40%, such as greater than about 50%, greater than about 60%, or even greater than about 70%.

The carrier material of the devices may have a surface area to weight ratio selected from about 20 m²/g to about 2000 m²/g, such as from about 20 m²/g to about 1000 m²/g, or even from about 100 m²/g to about 300 m²/g. In certain embodiments, the surface area is greater than about 200 m²/g, greater than about 250 m²/g or greater than about 300 m²/g. In certain embodiments, the surface area is about 200 m²/g.

In certain embodiments, the therapeutic agent is distributed to a pore depth from the surface of the material of at least about 10 microns, at least about 20 microns, at least about 30 microns, at least about 40 microns, at least about 50 microns, at least about 60 microns, at least about 70 microns, at least about 80 microns, at least about 90 microns, at least about 100 microns, at least about 110 microns, at least about 120 microns, at least about 130 micron, at least about 140 microns or at least about 150 microns. In certain embodiments, the therapeutic agent is distributed in the pores of the carrier material substantially uniformly.

The therapeutic agent may be loaded into the carrier material to a depth which is measured as a ratio of the depth to which the therapeutic agent penetrates the carrier material to the total width of the carrier material. In certain embodiments, the therapeutic agent is distributed to a depth of at least about 10% into the carrier material, to at least about 20% into the carrier material, at least about 30% into the carrier material, at least about 40% into the carrier material, at least about 50% into the carrier material, or at least about 60% into the carrier material.

Quantification of gross loading may be achieved by a number of analytic methods, for example, gravimetric, EDX (energy-dispersive analysis by x-rays), Fourier transform infra-red (FTIR) or Raman spectroscopy of the pharmaceutical composition or by UV spectrophotometry, titrimetric analysis, HPLC or mass spectroscopy of the eluted therapeutic agent in solution. Quantification of the uniformity of loading may be obtained by compositional techniques that are capable of spatial resolution such as cross-sectional EDX, Auger depth profiling, micro-Raman and micro-FTIR.

Porous silicon-based materials of the invention may be categorized by the average diameter of the pore size. Microporous silicon-based material has an average pore size less than 2 nm, mesoporous silicon-based material has an average pore size of between 2-50 nm and macroporous silicon-based material has a pore size of greater than 50 nm. In certain embodiments, greater than 50% of the pores of the silicon-based material have a pore size from 2-50 nm, greater than 60% of the pores of the silicon-based material have a pore size from 2-50 nm, greater than 70% of the pores of the silicon-based material have a pore size from 2-50 nm, greater than 80% of the pores of the silicon-based material have a pore size from 2-50 nm, or even greater than 90% of the pores of the silicon-based material have a pore size from 2-50 nm.

In certain embodiments, the carrier material comprises porous silicon dioxide, such as mesoporous silicon dioxide. In certain embodiments, the average pore size of the carrier material is selected from 2-50 nm, such as from about 5 to about 40 nm, from about 15 to about 40 nm, such as about 20 to about 30 nm. In certain embodiments, the average pore size is selected from about 2 to about 15 nm, such as about 5 to about 10 nm. In certain embodiments, the average pore size is about 30 nm.

In certain embodiments, the carrier material has a population of pores with a well-defined pore size, i.e., the distribution of pore sizes for the carrier material falls within a defined range. In certain embodiments, a well-defined population of pores has about 50% to about 99% of the pore sizes within about 1 nm to 15 nm of the average pore size for that population, preferably within about 10 nm, about 5 nm, or even within 3 nm or 2 nm of the average pore size for that population. In certain such embodiments, greater than about 50%, greater than about 60%, greater than about 70%, greater than about 80%, greater than about 90%, or even greater than about 95% of the pores of the carrier material have pore sizes within the specified range. Similarly, a population of pores with a well-defined pore size can be a population in which greater than about 50%, greater than about 60%, greater than about 70%, greater than about 80%, greater than about 90%, or even greater than about 95% of the pores have pore sizes within 20%, preferably within 15%, 10%, or even 5% of the average pore size for that population.

Pore (e.g., mesopore) size distribution can be quantified using established analytical methods such as gas adsorption, high resolution scanning electron microscopy, nuclear magnetic resonance cryoporosimetry and differential scanning calorimetry. In certain embodiments, more than one technique is used on a given sample.

Alternatively, a population of pores with a well-defined pore size can be a population for which the standard deviation of the pore sizes is less than 20%, preferably less than 15%, less than 10%, or even less than 5% of the average pore size for that population.

The pore size may be preselected to the dimensional characteristics of the therapeutic agent to control the release rate of the therapeutic agent in a biological system. Typically, pore sizes that are too small preclude loading of the therapeutic agent, while oversized pores do not interact with the therapeutic agent sufficiently strongly to exert the desired control over the rate of release. For example, the average pore diameter for a carrier material may be selected from larger pores, e.g., 15 nm to 40 nm, for high molecular weight molecules, e.g., 200,000-500,000 amu, and smaller pores, e.g., 2 nm to 10 nm, for molecules of a lower molecular weight, e.g., 10,000-50,0000 amu. For instance, average pore sizes of about 6 nm in diameter may be suitable for molecules of molecular weight around 14,000 to 15,000 amu, such as about 14,700 amu. Average pore sizes of about 10 nm in diameter may be selected for molecules of molecular weight around 45,000 to 50,000 amu, such as about 48,000 amu. Average pore sizes of about 25-30 nm in diameter may be selected for molecules of molecular weight around 150,000 amu.

The pore size may be preselected to be adapted to the molecular radius of the therapeutic agent to control the release rate of the therapeutic agent in a biological system. Molecular radii may be calculated by any suitable method such as by using the physical dimensions of the molecule based on the X-ray crystallography data or using the hydrodynamic radius which represents the solution state size of the molecule. As the solution state calculation is dependent upon the nature of the solution in which the calculation is made, it may be preferable for some measurements to use the physical dimensions of the molecule based on the X-ray crystallography data. As used herein the largest molecular radius reflects half of the largest dimension of the therapeutic agent.

In certain embodiments, the average pore diameter is selected to limit the aggregation of molecules, e.g., proteins, within a pore. It would be advantageous to prevent peptides, such as proteins, from aggregating in a carrier material as this is believed to impede the controlled release of molecules into a biological system. Therefore, a pore that, due to the relationship between its size and the size of a peptide, allows, for example, only one peptide to enter the pore at any one time will be preferable to a pore that allows multiple peptides to enter the pore together and aggregate within the pore. In certain embodiments, multiple peptides may be loaded into a pore, but due to the depth of the pore, the proteins distributed throughout this depth of the pore will aggregate to a lesser extent.

In certain embodiments, the carrier material comprises two or more different materials with different properties (e.g., pore sizes, particle diameters, or surface characteristics), each preselected to be adapted to a different therapeutic agent. For example, two different carrier materials may be admixed, one with a first population of pores whose pore size is adapted to a first therapeutic agent, the other with a second population of pores whose pore size is adapted to a second therapeutic agent. In certain other embodiments, the carrier material comprises a single material that has two or more well-defined populations of pores, e.g., wherein the carrier material is made by a molecular templating technique, wherein the characteristics of the pores are preselected for two or more therapeutic agents, e.g., two therapeutic agents with different molecular radii. Thus, the carrier material may deliver two or more therapeutic agents in the controlled manner described herein. In such embodiments, the loading of the therapeutic agents is preferably ordered from largest to smallest agent, so that the largest agent selectively adsorbs into the largest pores (i.e., it does not fit into the smaller pores), so that the larger pores do not adsorb smaller agents.

For example, if a carrier material comprises a first population of well-defined pores that are about 6 nm in diameter (i.e., suitable for molecules of molecular weight around 14,000 to 15,000 amu) and a second population of well-defined pores that are about 10 nm in diameter (i.e., suitable for molecules of molecular weight around 45,000 to 50,000 amu), the latter therapeutic agent (i.e., the one with molecules of molecular weight around 45,000 to 50,000 amu) is preferably added to the carrier material prior to adding the smaller therapeutic agent (i.e., the one with molecules of molecular weight around 14,000 to 15,000 amu). Alternatively and additionally, in the embodiment wherein composition comprises two different porous materials, each carrier material may be separately loaded with a different therapeutic agent and then the carrier materials may be combined to yield the composition.

In certain embodiments in which the carrier material has two or more distinct well-defined populations of pores (e.g., the distinct pore populations are substantially non-overlapping), the differences between the properties of the different populations of pores are preferably selected to limit the adsorption of each different therapeutic agent to a certain population of pores. In certain embodiments, the average pore size of the two or more distinct well-defined pore populations may be selected to limit the adsorption of the larger therapeutic agents into smaller pores. The average pore size differential may be defined as the difference between the average pore sizes for the different populations of pores in the carrier material. For example, an average pore size differential of at least 10 nm could indicate that the carrier material may comprise at least two populations of pores whose average pore sizes differ (“average pore size differential”) by at least 10 nm, e.g., the composition may comprise two pore populations having average pore sizes of 10 nm and 20 nm, three populations of pores with average pore sizes of 10 nm, 20 nm, and 30 nm, or four populations of pores with average pore sizes of 10 nm, 20 nm, 30 nm, and 40 nm. In certain embodiments, the average pore size differential is preferably at least about 5 nm, at least about 10 nm, at least 15 nm, at least about 20 nm, or at least about 30 nm. In certain embodiments, the two or more well-defined pore populations have distinct average pore sizes, such that the average pore sizes of any two populations differ by at least 20%, preferably at least 30%, 40%, or even 50% of the smaller average pore size.

In certain embodiments in which the carrier material has a non-uniform distribution of pore sizes, the carrier material has two or more well-defined populations of pores with distinct average pore sizes as described above. Similarly, a carrier material with a non-uniform distribution of pore sizes can be characterized as having a distribution of pore sizes having at least two local maxima (e.g., one at pore size equal to A and one at pore size equal to B), but as many as three or four local maxima, wherein the number of pores having the size of two adjacent local maxima (e.g., M_{XA} and M_{XB}) is at least three times, but preferably five times, ten times, or even 20 times the number of pores having a pore size that is the average of the pore sizes of the two local maxima (e.g., M_{NAB}, wherein the average of the pore sizes of the two local maxima is AV_{AB}). The distribution of pore sizes may also be described by the following equations, which also apply in certain embodiments wherein M_{XA} are M_{XB} are not equivalent, e.g., the distribution is not strictly bimodal:

M_{XA}≧3(M_{NAB}) and M_{XB}≧3(M_{NAB}),

wherein M_{XA}=# of particles of pore size A; M_{XB}=# of particles of pore size B; and M_{NAB}=# of particles of pore size (A+B)/2, and where the 3 may be replaced by any suitable multiplier as described above.

In certain embodiments, the therapeutic agent is selected from any agent useful in the treatment or prevention of diseases. In certain embodiments, the therapeutic agent is a biomolecule. Biomolecules, as used herein, refer to any molecule that is produced by a living organism, including large polymeric molecules such as proteins, polysaccharides, and nucleic acids as well as small molecules such as primary metabolites, secondary metabolites, and natural products or synthetic variations thereof. In certain embodiments, the therapeutic agent has a molecular weight between about 1,000 amu and about 10,000 amu, and maybe between about 1,000 amu and about 5,000 amu, between about 2,000 amu and about 5,000 amu, between about 3,000 amu and about 5,000 amu or between about 4,000 amu and about 5,000 amu. In some embodiment, the peptide can used in combination with any other agent useful in the treatment or prevention of diseases, or useful in diagnosis.

The size of a therapeutic agent may alternatively be characterized by the molecular radius, which may be determined, for example, through X-ray crystallographic analysis or by hydrodynamic radius. The therapeutic agent may be a peptide, e.g., with a molecular radius selected from 0.5 nm to 20 nm such as about 0.5 nm to 10 nm, even from about 1 to 8 nm. A therapeutic agent with molecular radius from 1 to 2.5 nm may be advantageously used with a carrier material with a minimum pore radius of from 4.5 to 5.8 nm. A therapeutic agent with a molecular radius of 7 nm may be advantageously used with a carrier material with a minimum pore radius of from 11 to 13 nm, such as about 12 nm. For example, insulin with a hydrodynamic radius of 1.3 nm may be used with a carrier material that has an average minimum pore radius of 4.8 nm. For example, cosyntropin (containing the first 24 amino acids of ACTH but retaining full function) has a calculated radius of 0.91 nm and may be used with a carrier material that has an average minimum pore radius of 4.4 nm

The protein-pore differential may be used to choose a suitable carrier material to accommodate the therapeutic agent. This calculation subtracts the molecular radius from the pore radius. Typically, the radius of the therapeutic agent would be the hydrodynamic radius or largest radius determined through x-ray crystallographic analysis. The pore radius would typically be the average pore radius of the carrier material. For example, the pore-protein differential for insulin, with a hydrodynamic radius of 1.3 nm and a pore with a minimum radius of 4.8 nm has a protein-pore differential of 3.5 nm. In certain embodiments, the protein-pore differential is selected from 3 to 6 nm, such as from 3.2 to 4.5 nm. The protein-pore differential may be about 3.2 nm, about 3.3 nm, about 3.4 nm, about 3.5 nm, about 3.6 nm, about 3.7 nm, about 3.8 nm, about 3.9 nm, about 4.0 nm, about 4.1 nm, about 4.2 nm, about 4.3 nm, about 4.4 nm or about 4.5 nm.

In certain embodiments, the walls of the carrier material that separate the pores have an average width of less than 5 nm, such as about 4.8 nm, about 4.6 nm, about 4.4 nm, about 4.2 nm, about 4.0 nm, about 3.8 nm, about 3.6 nm, about 3.4 nm, about 3.2 nm, about 3.0 nm, about 2.8 nm, or even about 2.6 nm. In certain embodiments, the walls of the carrier material that separate the pores have an average width of less than about 3 nm, such as about 2.8 nm, about 2.6 nm, about 2.4 nm, about 2.2 nm, about 2.0 nm, about 1.8 nm, about 1.6 nm, about 1.4 nm, about 1.2 nm, about 1.0 nm, or even about 0.8 nm.

Dimensionality and morphology of the carrier material particles can be measured, for example, by Transmission Electron Microscopy (TEM) using a 2000 JEOL electron microscope operating, for example, at 200 keV. Samples for TEM can be prepared by dispensing a large number of porous carrier materials onto a holey carbon film on a metal grid, via a dilute slurry.

In certain embodiments, the pores of the carrier material define space having a volume of about 0.1 mL/g to about 5 mL/g of the carrier material. In certain embodiments, the pore volume is about 0.2 mL/g to about 3 mL/g, such as about 0.4 mL/g to about 2.5 mL/g, such as about 1.0 mL/g to about 2.5 mL/g.

In certain embodiments, the load level of the carrier material is up to 70%, such as up to 40% by weight based on the combined weight of the carrier material and the therapeutic agent. The load level is calculated by dividing the weight of the loaded therapeutic agent by the combined weight of the loaded therapeutic agent and carrier material and multiplying by 100. In certain embodiments, the load level of the carrier material is greater than 1%, such as greater than 2%, greater than 3%, greater than 5%, greater than 10%, greater than 15%, greater than 20%, greater than 25%, greater than 30%, greater than 35%, greater than 40%, greater than 45% or greater than 50%. In certain embodiments, the load level of the carrier material is less than 5%, or between about 4% and about 6%. The load level may be between about 5% and about 10%. In certain embodiments, the load level of the carrier material is between about 10% and about 20%, between about 20% and about 30%, between about 30% and about 40%, between about 40% and about 50%, or between about 50% and about 60% by weight.

The load volume of the carrier materials described herein may be evaluated in terms of the volume of the pores in the porous material being occupied by the therapeutic agent. The percentage of the maximum loading capacity that is occupied by the therapeutic agent (that is, the percentage of the total volume of the pores in the porous carrier material that is occupied by the therapeutic agent) for carrier materials according to the invention may be from about 30% to about 100%, such as from about 50% to about 90%. For any given carrier material, this value may be determined by dividing the volume of the therapeutic agent taken up during loading by the void volume of the carrier material prior to loading and multiplied by one hundred.

In certain embodiments, the carrier materials of the invention are three-dimensional branched chain aggregates, formed by particles that collide, attach and sinter together. For example, fumed silica typically comprises small particles of silicon dioxide that can aggregate together to form larger particles.

In certain embodiments, the carrier materials of the invention are particles that, measured at the largest diameter, have an average size of about 1 to about 500 microns, such as about 5 to about 100 microns. In certain embodiments, a single particle measured at its largest diameter is about 1 to about 500 microns, such as about 5 to about 500 microns.

In order to increase the rate of loading of the particles of the invention, it may be advantageous to use relatively small particles. As smaller particles have pores with less depth for the therapeutic agent to penetrate, the amount of time needed to load the particles may be reduced. This may be particularly advantageous when the pore diameters are similar in dimensions to the molecular diameters or size of the therapeutic agents. Smaller particles may be from 1-20 microns, such as about 10-20 microns, e.g., about 15-20 microns, measured at the largest dimension.

In some aspects, greater than 60%, greater than 70%, greater than 80% or greater than 90% of the particles have a particle size of from 1-20 microns, preferably 5-15 microns, measured at the largest dimension. The particles may have an average particle size between 1 and 20 microns such as between 5-15 microns or about 15 microns, about 16 microns, about 17 microns, about 18 microns, about 19 microns.

Particle size distribution, including the mean particle diameter can be measured, for example, using a Malvern Particle Size Analyzer, Model Mastersizer, from Malvern Instruments, UK. A helium-neon gas laser beam may be projected through an optical cell containing a suspension of the carrier material. Light rays striking the carrier material are scattered through angles which are inversely proportional to the particle size. The photodetector array measures the light intensity at several predetermined angles and electrical signals proportional to the measured light flux values are then processed by a microcomputer system against a scatter pattern predicted from the refractive indices of the sample carrier material and aqueous dispersant.

Larger devices/implants are also envisioned for controlled delivery of therapeutic agents. The devices/implants of the invention may have an average size of about 1 mm to about 5 cm measured at the largest dimension. In certain embodiments, the devices/implants have an average size of about 5 mm to about 3 cm measured at the largest dimension. Particles greater than 1 mm, as measured at the largest dimension, may be useful for intramuscular, subcutaneous, intravitreal, or subdermal drug delivery.

In certain embodiments, the porous carrier materials described herein are used to stabilize sensitive therapeutic compounds, such as peptides. In certain embodiments, peptides that are partially or wholly unstable at elevated temperatures, such as room temperature or above, can be made stable at room temperature for prolonged periods of time. The peptides may be loaded into a carrier material such that an aqueous suspension of the peptide loaded into the carrier material is more stable than a corresponding aqueous solution of the peptide (i.e., an identical aqueous solution with and without the addition of the porous carrier material). For example, the peptide within the carrier material may have a half-life at room temperature (e.g., about 23° C.) that is greater than a half-life of the peptide without the carrier material under the same conditions. In certain embodiments, a peptide in the pores of the carrier material has a half-life that is at least twice as long as the peptide outside of the carrier material under the same conditions, more preferably, at least five times, at least 10 times, at least than 15 times, at least 20 times, at least 30 times, at least 40 times, at least 50 times, at least 60 times, or at least 100 times as long as the peptide outside of the carrier material.

Similarly, peptides may have a longer shelf life within the pores of the carrier material than in a corresponding aqueous solution, preferably at least twice as long, at least five times as long, at least 10 times as long, at least 20 times as long, at least 30 times as long, at least 40 times as long, at least 50 times as long, at least 60 times as long or at least 100 times as long.

In certain embodiments, peptides formulated as compositions comprising the carrier material and a peptide exhibit stability at the temperature of 25° C. for at least 15 days, or even about 1 month. Additionally or alternatively, in certain embodiments, the peptide-loaded compositions are stable at 25° C. for at least 6 months, at least 1 year, at least 1.5 years, at least 2 years, at least 2.5 years, at least 3 years or at least 4 years. Stability may be assessed, for example, by high performance size exclusion chromatography (HPSEC) or by comparing the biological activity of the stored peptide-loaded compositions against a sample of freshly prepared peptide-loaded devices or against the activity of the devices as measured prior to storage. Preferably, at the end of the storage period, the activity of the stored compositions is at least 75%, at least 80%, at least 85%, at least 90%, at least 95%, at least 98%, at least 99%, at least 99.5%, at least 99.8%, or even at least 99.9% of the activity of the corresponding freshly prepared compositions. Accordingly, the invention contemplates methods of treatment wherein peptide-loaded compositions are stored at 25° C. for at least 6 months, at least 1 year, at least 1.5 years, at least 2 years, at least 2.5 years, at least 3 years or at least 4 years prior to administering the compositions to a patient.

The invention further comprises methods of stabilizing peptides. Methods of the invention comprise loading peptide into the pores of the carrier material through any suitable method to form the compositions of the invention.

Methods of Preparation

The invention also provides methods of preparing silicon-based carrier materials. In certain embodiments, porous silicon-based carrier material may be prepared synthetically. For example, porous silica may be synthesized by reacting tetraethyl orthosilicate with a template made of micellar rods. In certain embodiments, the result is a collection of spheres or rods that are filled with a regular arrangement of pores. The template can then be removed, for example, by washing with a solvent adjusted to the proper pH. In certain embodiments, the porous silicon-based carrier material may be prepared using a sol-gel method or a spray drying method. In certain embodiments, the preparation of the carrier material involves one or more techniques suitable for preparing porous silicon-based material. In some embodiments, the method of making the compositions comprises introducing the therapeutic agent into the mesopores of the carrier material. In some embodiments, the method of making the sustained release drug delivery composition comprises preparing the carrier material comprising a resorbable or bioerodible mesoporous silicon-based compound by providing a body comprising semiconductor silicon and treating the semiconductor silicon to make at least a portion of the body porous prior to introducing the therapeutic agent.

In some embodiments, the method of administering at least one therapeutic agent to a mammal in need thereof comprises administering the sustained release drug delivery composition. In some embodiments, the method of administering at least one therapeutic agent to a mammal comprises administering the at least one therapeutic agent is present in the pores of the carrier material. In some embodiment, the method of administering at least one therapeutic agent to a mammal comprises delivering the at least one therapeutic agent to a specific site of the mammal. In some embodiments, the method of administering at least one therapeutic agent to a mammal comprises delivering a peptide selected from adrenocorticotropic hormone (ACTH) or its analogs. In some embodiments, the method of administering at least one therapeutic agent to a mammal comprises delivering adrenocorticotropic hormone (ACTH) analogs selected from corticotropin, tetracosactide or cosyntropin. In some embodiments, the method of administering at least one therapeutic agent to a mammal comprises releasing at a release rate that depends at least in part upon the rate of release of the agent from the pores of the carrier material. In some embodiments, the method of administering at least one therapeutic agent to a mammal comprises releasing at a release rate that depends at least in part on the rate of resorption or bio-erosion of the carrier material.

Pores may be introduced to the silicon-based carrier material through techniques such as anodization, stain etching, or electrochemical etching. In an exemplary embodiment, anodization employs a platinum cathode and silicon wafer anode immersed in hydrogen fluoride (HF) electrolyte. Corrosion of the anode producing pores in the material is produced by running electrical current through the cell. In particular embodiments, the running of constant direct current (DC) is usually implemented to ensure steady tip-concentration of HF resulting in a more homogeneous porosity layer.

In certain embodiments, pores are introduced to the silicon-based carrier material through stain-etching with hydrofluoric acid, nitric acid and water. In certain embodiments, a combination of one or more stain-etching reagents is used, such as hydrofluoric acid and nitric acid. In certain embodiments, a solution of hydrofluoric acid and nitric acid are used to form pores in the silicon-based material.

The porosity of the material can be determined by weight measurement. BET analysis may be used to determine any one or more of the pore volume, pore size, pore size distribution and surface area of the carrier material. BET theory, named after the combined surname initials of authors of the theory, applies to the physical adsorption of gas molecules on a solid surface and serves as the basis for an important analysis technique for the measurement of the specific surface area of a material (J. Am. Chem. Soc., v. 60, p. 309 (1938)). The BET analysis may be performed, for example, with a Micromeritics ASAP 2000 instrument available from Micromeritics Instrument Corporation, Norcross, Ga. In an exemplary procedure, the sample of carrier material may be outgassed under vacuum at temperatures, for example, greater than 200° C. for a period of time such as about 2 hours or more before the measurements are taken. In certain embodiments, the pore size distribution curve is derived from the analysis of the adsorption branch of the isotherm output. The pore volume may be collected at the P/P₀=0.985 single point.

One or more drying techniques may be used in the preparation of porous silicon-based materials of the invention. For example, to prevent cracking of the porous silicon-based material, the material may be dried by supercritical drying, freeze drying, pentane drying or slow evaporation. Supercritical drying involves superheating the liquid pore above the critical point to avoid interfacial tension. Freeze drying involves freezing and subliming any solvents under vacuum. Pentane drying uses pentane as the drying liquid instead of water and as a result may reduce capillary stress due to the lower surface tension. Slow evaporating is a technique which can be implemented following the water or ethanol rinsing and may be effective at decreasing the trap density of solvent within the material.

The surface of the porous silicon-based material may be modified to exhibit properties such as improved stability, cell adhesion or biocompatibility. Optionally, the material may be exposed to oxidizing conditions such as through thermal oxidation. In an exemplary embodiment, the process of thermal oxidation involves heating the silicon-based material to a temperature above 1000° C. to promote full oxidation of the silicon-based material. Alternatively, the surface of the carrier material may be oxidized so that the carrier material comprises a framework of elemental silicon partially, substantially or fully covered by an oxidized surface such as a silicon dioxide surface.

The surface of the porous silicon-based material or a portion thereof may be derivatized. In an exemplary embodiment, the surface of a porous silicon-based material may be derivatized with organic groups such as alkanes or alkenes. In a particular embodiment, the surface of the carrier material may be derivatized by hydrosilation of silicon. In particular embodiments, the derivatized carrier materials may function as biomaterials, incorporating into living tissue.

Any one or more of electrostatic interactions, capillary action and hydrophobic interactions may enable loading of the therapeutic agent into the pores of the carrier material. In certain embodiments, the carrier material and therapeutic molecules are placed in a solution and the peptides are drawn from the solution into the pores of the carrier material, reminiscent of a molecular sieve's ability to draw water from an organic liquid. Hydrophobic drugs may be better suited for loading into carrier materials that are predominantly formed from silicon (e.g., greater than 50% of the material is silicon) while hydrophilic drugs may be better suited for loading into a carrier material that is characterized as mostly silica (e.g., greater than 50% of the carrier material is silica). In certain embodiments, the loading of peptides into the pores of the carrier material is driven by external factors such as sonication or heat. The carrier material, or portion thereof, may have an electrostatic charge and/or the therapeutic agent, or portion thereof, may have an electrostatic charge. Preferably, the carrier material, or portion thereof, has the opposite electrostatic charge as the therapeutic agent, or portion thereof, such that adsorption of the therapeutic agent into the pores of the carrier material is facilitated by the attractive electrostatic forces. In certain embodiments, the therapeutic agent or the carrier material may not have an electrostatic charge by itself, but is instead polarizable and has its polarity modified in the proximity of the carrier material or the therapeutic agent, respectively, which facilitates the adsorption of the therapeutic agent in the pores of the carrier material.

For example, in the body, at physiological pH, silicon dioxide, such as mesoporous silicon dioxide or amorphous silica, exhibits a negatively charged surface, which promotes electrostatic adsorption of positively charged peptides. ACTH and its synthetic analogs, such as cosyntropin, engage in this kind of electrostatic interactions because of the positively charged Lys(15)-Lys(16)-Arg(17)-Arg(18) sequence in their structures.

The carrier material may comprise a coating or surface modification to attract the therapeutic agent into the pores. In certain embodiments, the carrier material is coated or modified in whole or in part with a material comprising moieties that are charged in order to attract a peptide into the pores of the carrier material. In other embodiments, the moieties may be appended directly to the carrier material. For example, amine groups may be covalently appended onto the surface of the carrier material such that when protonated at physiological pH, the surface of the carrier material carries a positive charge, thereby, for example, attracting a peptide with a negatively charged surface. In other embodiments, the carrier material may be modified with carboxylic acid moieties such that when deprotonated at physiological pH, the carrier material carries a negative charge, thereby attracting proteins or antibodies with positively charged surfaces into the pores.

In certain embodiments, the carrier material may be a material other than porous silica. Although silicon-based materials are preferred carrier materials for use in the present invention, additional bioerodible materials with certain properties (e.g. porosity, pore size, particle size, surface characteristics, bioerodibility, and resorbability) in common with the silicon-based materials described herein may be used in the present invention. Examples of additional materials that may be used as carrier materials are bioerodible ceramics, bioerodible metal oxides, bioerodible semiconductors, bone phosphate, phosphates of calcium (e.g. hydroxyapatite), other inorganic phosphates, porous carbon black, carbonates, sulfates, aluminates, borates, aluminosilicates, magnesium oxide, calcium oxide, iron oxides, zirconium oxides, titanium oxides, and other comparable materials. Many of these porous materials can be prepared using techniques (e.g., templating, oxidation, drying, and surface modification) that are analogous to the aforementioned techniques used to prepare porous silicon-based carrier materials.

In certain embodiments, the therapeutic agent may be incorporated into the carrier material following complete formation of the carrier material. Alternatively, the therapeutic agent may be incorporated into the carrier material at one or more stages of preparation of the carrier material. For example, the therapeutic agent may be introduced to the carrier material prior to a drying stage of the carrier material, or after the drying of the carrier material or at both stages. In certain embodiments, the therapeutic agent may be introduced to the carrier material following a thermal oxidation step of the carrier material. In certain aspects, the therapeutic agent is introduced as the final step in the preparation of the compositions.

More than one therapeutic agent may be incorporated into drug delivery composition. In certain such embodiments, each therapeutic agent may be a peptide. For example, an ocular delivery vehicle composition may be impregnated with two therapeutic agents for the treatment of glaucoma, or one therapeutic agent for the treatment of macular degeneration and another agent for the treatment of glaucoma. Alternatively, more than one therapeutic agent may be incorporated into a plurality of compositions. For example, two ocular delivery vehicle compositions may be impregnated with a therapeutic agent for the treatment of glaucoma, wherein one delivery vehicle composition is administered at the back of the eye and the other is administered at the front of the eye.

In certain aspects, e.g., when both small molecule therapeutic agents and larger molecular therapeutic agents such as proteins are incorporated into a composition, the therapeutic agents may be incorporated into the carrier material at different stages of the preparation of the composition. For example, a small molecule therapy may be introduced into the carrier material prior to an oxidation or drying step and a large molecule therapeutic agent may be incorporated following an oxidation or drying step. Similarly, multiple different therapeutic agents of the same or different types may be introduced into a finished carrier material in different orders or essentially simultaneously. When a carrier material comprises a single material, or combination of multiple materials with multiple pore sizes, the larger therapeutic agent is preferably added to the carrier material prior to adding the smaller therapeutic agent to avoid filling the larger pores with the smaller therapeutic agent and interfering with adsorption of the larger therapeutic agent. For example, if a carrier material comprises a single material, or combination of multiple materials, that has some well-defined pores that are about 6 nm in diameter (i.e., suitable for molecules of molecular weight around 14,000 to 15,000 amu) and some well-defined pores that are about 10 nm in diameter (i.e., suitable for molecules of molecular weight around 45,000 to 50,000 amu), the latter therapeutic agent (i.e., the one with molecules of molecular weight around 45,000 to 50,000 amu) are preferably added to the carrier material prior to adding the smaller therapeutic agent (i.e., the one with molecules of molecular weight around 14,000 to 15,000 amu). Alternatively and additionally, in the embodiment wherein the two different porous materials together comprise the composition, each carrier material may be separately loaded with a different therapeutic agent and then the carrier materials may be combined to yield the composition.

The therapeutic agent may be introduced into the carrier material in admixture or solution with one or more pharmaceutically acceptable excipients. The therapeutic agent may be formulated for administration in any suitable manner, suitably for subcutaneous, intramuscular, intraperitoneal or epidermal introduction or for implantation into an organ (such as the eye, liver, lung or kidney). Therapeutic agents according to the invention may be formulated for parenteral administration in the form of an injection, e.g., intraocularly, intravenously, intravascularly, subcutaneously, intramuscularly or infusion, or for oral administration.

In certain embodiments, the porous silicon-based carrier material is loaded with the one or more therapeutic agents at the point of service, such as in the doctor's office or hospital, prior to administration of the carrier material. For example, the porous silicon carrier material may be loaded with the therapeutic agent a short period of time prior to administration, such as 24 hours or less prior to administration, 3 hours or less prior to administration, 2 hours or less prior to administration, 1 hour or less prior to administration or 30 minutes or less prior to administration.

The carrier material may be in any suitable form prior to loading with the therapeutic agent such as in the form of a dry powder or particulate or formulated in an aqueous slurry, e.g., with a buffer solution or other pharmaceutically acceptable liquid. The therapeutic agent may be in any suitable form prior to loading into the carrier material such as in a solution, slurry, or solid such as a lyophilisate. The carrier material and/or the therapeutic agent may be formulated with other components such as excipients, preservatives, stabilizers, or therapeutic agents, e.g., antibiotic agents.

In some embodiments, the carrier material may be formulated (and packaged and/or distributed) already loaded with peptides, while in other embodiments, the carrier material or carrier material formulation is formulated (and packaged and/or distributed) essentially free of peptides, e.g., contains less than 5% peptides or less than 2% peptides, e.g., for combination with a peptide at the time of administration.

In some embodiments, the therapeutic agent may be formulated (and packaged and/or distributed) in combination with a carrier material as described above to provide a solution, suspension, or slurry with a concentration of >50 mg/mL, such as >60 mg/mL, such as >75 mg/mL of the agent. In some embodiments, the therapeutic agent may be formulated (and packaged and/or distributed) with a surfactant in combination with a carrier material as described above, wherein the therapeutic agent has a maximum concentration equal to or less than 50 mg/mL. In some embodiments, a peptide agent may be formulated (and packaged and/or distributed) in combination with a carrier material as described above to provide a composition with a concentration of >0.1 mg/mL, >0.2 mg/mL, >0.25 mg/mL, >0.5 mg/mL, >1 mg/mL, >10 mg/mL, >15 mg/mL or >20 mg/mL of the peptide agent.

The therapeutic agent may be formulated (and packaged and/or distributed) with stabilizers, excipients, surfactants or preservatives. In particular embodiments, therapeutic agent is formulated (and packaged and/or distributed) essentially free of any one or more of stabilizers, excipients, surfactants and preservatives, e.g., contains less than 1 mg/mL or preferably less than 0.1 mg/mL of a stabilizer, excipients, surfactant or preservative. The formulation of the therapeutic agent may contain less than 1 mg/mL of surfactants such as less than 0.1 mg/mL of surfactants.

In certain embodiments, the carrier material may be sold and/or distributed preloaded in any portion of a syringe such as the barrel of a syringe or the needle of a syringe, in any suitable form, such as a dry powder or particulate, or as a slurry (e.g., in combination with a biocompatible liquid, such as an aqueous solution). The preloaded syringe may comprise other components in addition to the carrier material such as excipients, preservatives, therapeutic agents, e.g., antibiotic agents or stabilizers. The preloaded syringe may include biomolecules, such as peptides, or may comprise a solution that is essentially free of biomolecules, e.g., less than 5% biomolecules or less than 2% biomolecules.

In certain embodiments, the porous silicon-based carrier material is loaded with one or more therapeutic agents within the barrel of a syringe. In particular embodiments, the carrier material is located within the barrel of a syringe as discussed above or it may be drawn up into a syringe from a separate vessel. With the carrier material in the syringe, a solution containing one or more therapeutic agents may be drawn into the syringe, thereby contacting the carrier material. Alternatively, the carrier material may be drawn up into the syringe after the therapeutic agent or a solution thereof is drawn into the barrel of the syringe. Once these components are combined, the mixture is allowed to incubate for a period of time to allow the therapeutic agent to load into the pores of the carrier material. In certain embodiments, the mixture is incubated for about 3 hours or less, about 2 hours or less, or about 1 hour or less, e.g., for about 30 minutes, about 20 minutes, about 10 minutes or about 5 minutes.

In certain embodiments, the composition, such as a particle, may comprise a coating to regulate release of the therapeutic agent. For example, the device may be coated with an excipient to obtain a desired release profile of the therapeutic agent from the composition.

Methods of Use

In certain embodiments, the compositions are used to prevent or treat a condition of a patient. The various embodiments provided herein are generally provided to deliver a therapeutically effective amount of a therapeutic agent locally, i.e., to the site of the pain, disease, etc., in a patient. In certain embodiments, the compositions of the invention may be delivered to any site on the surface or within the body of a patient. For example, compositions of the invention may be used on the surface of the skin or eye or may be implanted under the skin, within a muscle, within an organ, adjacent to a bone, within the eye or at any other location where controlled release of a therapeutic agent would be beneficial. The compositions may be administered intravitreally, subcutaneously, subconjunctivally, intraperitoneally, intramuscularly or subretinally. In certain embodiments, the compositions of the invention are delivered to the surface of the eye or within the eye such as within the uveal tract of the eye or within the vitreous of the eye.

In certain embodiments, the compositions of the invention are used to treat intraocular diseases, such as back of the eye diseases. Exemplary intraocular diseases include iritis, iridocyclitis, diffuse posterior uveitis and choroiditis; optic neuritis; chorioretinitis; anterior segment inflammation. Other examples of intraocular diseases include glaucoma, age-related macular degeneration, such as wet age-related macular degeneration, diabetic macular edema, geographic atrophy, choroidal neovascularization, uveitis, diabetic retinopathy, retinovascular disease and other types of retinal degenerations.

In certain embodiments, the compositions of the invention are used to treat diseases on the surface of the eye. Exemplary diseases include viral keratitis and chronic allergic conjunctivitis.

In certain embodiments, the method for treating an ocular condition comprises disposing the compositions on the surface of the eye or within the eye such as within the vitreous or aqueous of the eye. In certain embodiments, the compositions are injected or surgically inserted within the eye of the patient. In certain embodiments, the compositions are injected within the eye of the patient, e.g., into the vitreous of the eye. In certain embodiments, the compositions are injected as a composition. In certain embodiments, a composition comprises multiple particles. The composition may comprise particles with an average size between about 1 micron to about 500 microns. In certain embodiments, the composition comprises particles with an average particle size between 5 microns and 300 microns, such as between about 5 microns and 100 microns.

In certain embodiments, the invention comprises a method of loading a therapeutic agent into the porous silicon-based carrier material prior to administration to a patient, such as shortly before administration to a patient. A healthcare practitioner may obtain the therapeutic agent or agents and the silicon-based carrier material, for example, together in a package as part of a kit or separately. The therapeutic agent or agents may be obtained in solution such as an aqueous or organic solution, as a lyophilisate for reconstitution, or in any other suitable form.

The practitioner may introduce the therapeutic agent or agents to the carrier material in any suitable manner, such as by incubation of the agent and the carrier material in a vial or in the barrel of a syringe, trocar or needle. In particular embodiments, where the therapeutic agent is loaded onto the carrier material in a vial, the carrier material may be incubated with the therapeutic agent or agents or a solution thereof in the vial for a period of time, such as less than 24 hours, less than 2 hours, less than 1 hour, or even about 30 minutes or less.

In other embodiments, the carrier material is preloaded in the barrel of a syringe and the therapeutic agent or agents or a solution thereof is drawn into the syringe, forming a mixture with the carrier material. The mixture in the syringe may be allowed to incubate for a period of time such as 30 minutes or less. In certain embodiments, the particles are sterilized at one or more stages during the preparation of the carrier material, e.g., immediately prior to administration or prior to loading the syringe. In certain embodiments, any suitable method for sterilizing the carrier material may be used in preparation for implantation.

In certain aspects, compositions of the invention may be used to administer any therapeutic agent in a sustained fashion to a patient in need thereof. The compositions of the invention are not limited to ocular and intraocular use and may be used in any part of the body. For example, compositions of the invention may be used to administer therapeutic agents subdermally similar to the Norplant contraceptive device. In other embodiments, compositions of the invention are used to administer biomolecules over a sustained period of time for the treatment of chronic diseases such as arthritis. The compositions of the invention may be located any place in the body such as within a muscle. The compositions may comprise multiple small particles such as multiple particles 500 microns or less. The compositions may comprise larger particles such as greater than 500 microns or one or more particles greater than 1 mm in size such as greater than 10 mm.

The method of administering a therapeutic agent may comprise: a. providing a syringe preloaded with a porous silicon-based carrier material; b. contacting the carrier material with a therapeutic agent; and c. administering the carrier material to the patent. The porous silicon-based carrier material may be preloaded in any portion of the syringe such as the barrel of the syringe, an insert between the needle and the barrel, or in the needle of the syringe. The porous material may be preloaded into a portion of the syringe which may be removably coupled to other portions of a syringe, e.g., a cartridge. For example, the porous silicon material may be preloaded in an insert that can be removably attached between the barrel and the needle of a syringe wherein the remainder of the syringe parts are chosen from any commercially available syringe parts. In such embodiments, the insert may include one or more filters to prevent the particles from leaving the insert, such as a filter proximal to the point of attachment of the barrel with the porous carrier material positioned between the filter and the syringe needle. The filter may serve to contain the carrier material while being contacted with the therapeutic agent for loading the therapeutic agent into the pores of the carrier material. The filter may then be removed, reversed, bypassed or avoided so as to administer the loaded carrier material to the patient.

The porous silicon-based material may be preloaded into the needle of a syringe, the openings of which may be blocked by one or more disengageable blocks or filters that prevent the particles from exiting the needle until such time as is desired. Either before or after the carrier material has been loaded with the therapeutic agent, the block may be disengaged so as to permit administration of the loaded carrier material to the patient, e.g., through the needle. The preloaded needle may be removably coupled to any commercially available syringe barrel or may be affixed to a syringe barrel.

Step b of the method for administering a therapeutic agent described may be carried out by drawing the therapeutic agent into the syringe, such as by drawing the therapeutic agent in a mixture or solution into the syringe barrel. The therapeutic agent may be a peptide. The therapeutic agent may be released to the patient over the course of up to four, six, or even up to twelve months after administration. In some embodiments, the therapeutic agent is released to the patient over the course of 1 month to 6 months. In preferred embodiments, the therapeutic agent is released to the patient over the course of 2 days to 2 weeks. In preferred embodiments, the therapeutic agent is released to the patient over the course of 4 days to 12 days. In preferred embodiments, the therapeutic agent is released to the patient over the course of 6 days to 10 days.

In certain embodiments, the carrier material is loaded in vivo by separately administering the carrier material and therapeutic agent to the patient. First, either the carrier material or a therapeutic agent, or a formulation containing the carrier material or a therapeutic agent, is administered to a patient. Second, the carrier material or a therapeutic agent, or a formulation containing the carrier material or a therapeutic agent, whichever was not delivered in the first step, is administered to the same site of the patient, allowing the therapeutic agent to adsorb into the pores of the carrier material. The adsorption of the therapeutic agent in the pores of the carrier material takes place over the first minutes, hours, or days after the second step, until the adsorption of the therapeutic agent in the pores of the carrier material reaches an equilibrium with the desorption of the agent from the carrier material into the surrounding environment, e.g., on the surface or within the body of a patient. Thereafter, the composition may release a therapeutically effective amount of the therapeutic agent over a time period that is longer than the initial re-equilibration time period, e.g., hours, days, weeks, months, or years.

Exemplary routes of administration that can be used include oral, buccal, parenteral, intravenous, intra-arterial, subcutaneous, intramuscular, topical, intracranial, intraorbital, ophthalmic, intraventricular, intracapsular, intraspinal, intracisternal, intraperitoneal, intranasal, aerosol, or administration by suppository. In certain embodiments, the composition is injected or surgically inserted subcutaneously. In certain embodiments, the device is delivered to the patient intravenously, or intraarticularly. In certain embodiments, the composition is delivered buccally. In certain embodiments, the composition is delivered rectally.

In some embodiments, the composition is administered orally. Oral administration can be used, for instance, to deliver active agents to the stomach, small intestine, or large intestine. Formulations for oral administration may be in the form of capsules, cachets, pills, tablets, lozenges (using a flavored basis, usually sucrose and acacia or tragacanth), powders, granules, and the like, each containing a predetermined amount of an active ingredient. Solid dosage forms for oral administration (capsules, tablets, pills, dragees, powders, granules, and the like), may comprise the carrier material and one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. The oral compositions can also include sweetening, flavoring, perfuming, and preservative agents.

In certain embodiments, multiple carrier materials are delivered to the patient such as two carrier materials, three carrier materials, four carrier materials or five carrier materials or more. The carrier materials may be substantially identical in size or composition or may have different sizes, a make up of different carrier materials or be loaded with different therapeutic agents. The multiple carrier materials may be administered to the patient simultaneously or over a period of time, and at one or more locations of the patient's body.

In certain embodiments, the therapeutic agent is released from the composition into the surrounding biological system over a duration of days, weeks, months or years. In certain such embodiments, the therapeutic agent is released over a course of time selected from one day to two years, such as from two weeks to about one year, such as about one month to about one year. The composition may release the drug into the eye over the course of 1 day to 12 months, such as 1 day to 6 months, such as over the course of 1 week to 3 months. In certain embodiments, the therapeutic agent is released within two years, such as with 18 months, within 15 months, within one year, within 6 months, within three months, or even within two months. In certain embodiments, the release of the therapeutic agent from the composition occurs in a controlled manner such that a large percentage of the total impregnated therapeutic agent is not released immediately or within a short time span, e.g., within minutes or hours of administration. For example if the desired drug delivery time is 2 months, the total impregnated therapeutic agent may, for example, be released at a rate of approximately 1/60th of the impregnated therapeutic agent per day. In certain embodiments, controlled release involves the release of a therapeutic agent over the course of, for example, 1 month, 2 months, 3 months, 4 months, 5 months, 6 months, 7 months, or 8 months, wherein the amount of the agent released charts linearly with respect to the full course of delivery. In some embodiments, there may be a burst effect of the therapeutic agent shortly after administration, followed by a substantially constant release over a subsequent period of time. The burst effect may last, for example, from 1-10 days during which a percentage of the loaded drug is released. After the burst, the remainder of the therapeutic agent may be released constantly over a certain period of time. For example, in certain embodiments, less than 10% of the therapeutic agent is released over the first day following administration, and a further 50% is constantly released over the subsequent 2-30 days, e.g., at a substantially constant rate of release. In another exemplary embodiment, less than 10% of the therapeutic agent is released in the first 5 days following administration, followed by constant release of 50% of the therapeutic agent over the subsequent 25 days. By substantially constant release, it is meant that the rate of release of the therapeutic agent from the composition is essentially constant over a certain period of time.

In certain embodiments, the therapeutic agent begins being released immediately after being administered. In certain embodiments, the therapeutic agent is released over the course of approximately 3 to 8 months, such as over the course of about 6 months. In certain embodiments, additional compositions of the invention are administered to a patient at appropriate periods to ensure a substantially continuous therapeutic effect. For example, successive doses of a composition that releases a drug for a period of six months may be administered biannually, i.e., once every six months.

Pharmacokinetics may be determined by serum and vitreous analyses using ELISA.

In certain embodiments, the carrier material may completely or partially bioerode within a biological system. In certain embodiments, the carrier material may be resorbed by the biological system. In certain embodiments, the carrier material may be both bioerodible and resorbable in the biological system. In certain embodiments, the carrier material may be partially bioactive such that the material incorporates into living tissue. In some embodiments, after implantation, the carrier material does not substantially mineralize or attract mineral deposits. For instance, in some embodiments, the carrier material does not substantially calcify when placed in situ in a site where calcification is undesirable.

In certain embodiments, the carrier material may bioerode in a biological system. In certain embodiments, greater than about 80% of the carrier material will bioerode in a biological system, such as greater than about 85%, greater than about 90%, greater than about 92%, greater than about 95%, greater than about 96%, greater than about 97%, greater than about 98%, greater than about 99%, greater than 99.5%, or even greater than 99.9%. In certain embodiments, where the carrier material bioerodes, it is partially or completely resorbed.

In certain embodiments, the carrier material may substantially bioerode over the course of 1 week to 3 years. In certain embodiments, substantial bioerosion refers to erosion of greater than 95% of the carrier material. In certain embodiments, substantial bioerosion occurs over the course of about 1 month to about 2 years, such as about 3 months to 1 year. In certain embodiments, substantial bioerosion occurs within about 3 years, such as within about 2 years, within about 21 months, within about 18 months, within about 15 months, within about 1 year, within about 11 months, within about 10 months, within about 9 months, within about 8 months, within about 7 months, within about 6 months, within about 5 months, within about 4 months, within about 3 months, within about 2 months, within about 1 month, within about 3 weeks, within about 2 weeks, within about 1 week, or even within about 3 days. In certain embodiments, where the carrier material bioerodes, it is partially or completely resorbed.

In certain embodiments, the extent of bioerosion may be evaluated by any suitable technique used in the art. In exemplary embodiments, the bioerosion is evaluated through an in vitro assay to identify degradation products or in vivo histology and analysis. The biodegradability kinetics of the porous carrier material may be assessed in vitro by analyzing the concentration of the principle degradation product in the relevant body fluid. For porous silicon-based carrier materials in the back of the eye, for example, the degradation product may include orthosilicic acid, quantified, for example, by the molybdate blue assay, and the body fluid may be simulated or real vitreous humor. The biodegradability kinetics in vivo may be determined by implanting a known quantity of the porous silicon-based material into the relevant body site and monitoring its persistence over time using histology combined with, for example, standard microanalytical techniques.

The invention now being generally described, it will be more readily understood by reference to the following examples which are included merely for purposes of illustration of certain aspects and embodiments of the present invention, and are not intended to limit the invention.

### EXAMPLES

### Materials

### Specifications of Commercial Porous Silica

| | | | Surface | Pore |
|---|---|---|---|---|
| | | Nominal Pore Size | Area | Volume |
| Supplier | Trade Name | (Å) | (m²/g) | (mL/g) |
| Grace Davison | Davisil | 60 | 550 | 0.9 |
| Discovery | | 150 | 330 | 1.2 |
| Sciences | | 250 | 285 | 1.8 |
| | | 500 | 80 | 1.1 |
| | | 1000 | 40 | 1.1 |
| SiliCycle | SiliaSphere PC | 300 | 100 | 1.1 |
| Cabot | Cab-O-Sil | — | 200 | — |
| Corporation | | | | |

### Example 1

Oxidized anodized porous silicon particles were added to a solution of ACTH in PBS to load the ACTH into the particles (carrier:ACTH 10:1 w:w). The supernatant was removed after 30 minutes and fresh buffer was added to the drug-loaded particles. The in vitro release rate test was conducted in PBS at 37° C. The release medium was replaced daily and the drug release from the particles was quantitatively measured by HPLC over 7 to 10 days (FIG. 1).

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

While the above described embodiments are in some cases described in terms of preferred characteristics (e.g., preferred ranges of the amount of effective agent, and preferred thicknesses of the preferred layers) these preferences are by no means meant to limit the invention. As would be readily understood by one skilled in the art, the preferred characteristics depend on the method of administration, the beneficial substance used, the shell and carrier materials used, the desired release rate and the like.

All of the foregoing U.S. patents and other publications are expressly incorporated by reference herein in each of their entireties.

1. A sustained release drug delivery composition comprising:
a) a porous carrier material comprising a silicon-based compound; and
b) at least one therapeutic agent associated with the carrier material, wherein the at least one therapeutic agent includes adrenocorticotropic hormone (ACTH) or an analog thereof.

2. The delivery composition according to claim 1, wherein the silicon-based compound comprises one or more of: porous silicon, polycrystalline silicon, and resorbable or bio-erodible silicon.

3. The delivery composition according to claim 2, wherein the porous silicon is mesoporous silicon.

4. The delivery composition according to any preceding claim, wherein the silicon-based compound has a silica or silicon oxide surface.

5. The delivery composition according to claim 1, wherein the silicon-based compound is amorphous silica.

6. The delivery composition according to any preceding claim, wherein the at least one therapeutic agent includes an ACTH analog selected from corticotropin, tetracosactide or cosyntropin.

7. The delivery composition according to any preceding claim, wherein the carrier material is sized for injection through a needle.

8. A method of making the delivery composition according to claim 2, comprising introducing the therapeutic agent into the pores of the carrier material.

9. A method of administering at least one therapeutic agent to a mammal in need thereof, comprising administering a composition according to any one of claims 1-7 to a mammal.

10. The method according to claim 9, wherein the at least one therapeutic agent is adsorbed to a surface of the carrier material.

11. The method according to claim 9 or 10, wherein the composition delivers the at least one therapeutic agent locally to a specific site of the mammal.

12. The method according to claim 9, 10, or 11, wherein the at least one therapeutic agent includes an ACTH analog selected from corticotropin, tetracosactide or, cosyntropin.
